(19) United States
(12) Patent Application Publication
(10) Pub. No.: US 2008/0082038 A1
(43) Pub. Date: Apr. 3, 2008

(54) Medical Device including a Bioactive in a Non-ionic and an Ionic Form and Methods of Preparation Thereof

(75) Inventors: Michael T. V. Johnson, Willow Creek, MT (US); Larry A. Alegria, Bozeman, MT (US); Benjamin P. Luchsinger, Bozeman, MT (US)

Correspondence Address: BRINKS HOFER GILSON & LIONE/CHICAGO/COOK, PO BOX 10395, CHICAGO, IL, 60610, US

(73) Assignee: Vance Products Incorporated, d/b/a/ Cook Urological Incorporated, Spencer, IN (US)

(21) Appl. No.: 11/864,360

(22) Filed: Sep. 28, 2007

Related U.S. Application Data
(60) Provisional application No. 60/847,875, filed on Sep. 28, 2006.
(60) Provisional application No. 60/857,626, filed on Nov. 8, 2006.

(51) Int. Cl. A61B 17/60; A61B 17/00; A61F 2/00; A61F 2/04; A61K 31/14; A61K 31/245; A61K 31/4025; A61K 31/496; A61K 31/5375; A61M 25/00; A61M 16/00; A61K 38/02; A61K 38/00; A61K 31/727; A61K 31/7048; A61K 31/7036; A61K 31/65; A61K 31/505; A61K 31/445; A61K 31/357; A61K 31/167; A61F 2/82; A61F 2/02; A61B 17/03

(52) U.S. Cl. 604/8; 128/207.14; 424/422; 514/152; 514/2; 514/239.2; 514/254.11; 514/275; 514/29; 514/330; 514/40; 514/422; 514/452; 514/535; 514/54; 514/626; 514/643; 514/8; 604/264; 604/523; 606/151; 606/59; 606/70; 623/23.7

(57) ABSTRACT

A medical device having a bioactive in a salt and a free-base form contained within the device and/or coated onto a surface of the device. The salt and the free-base form have different solubilities in an aqueous medium. The elution of bioactive from the device in an aqueous medium is tailored by fixing the ratio of forms. Methods of preparing such a device are also provided.

### RELATED APPLICATIONS

This application claims the benefit of the filing date under 35 U.S.C. §119(e) of Provisional U.S. Patent Application Ser. Nos. 60/847,875, filed Sep. 28, 2006, and 60/857,626, filed Nov. 8, 2006, the contents of which are hereby incorporated by reference.

### TECHNICAL FIELD

The present embodiments relate generally to medical devices including a bioactive in a non-ionic and an ionic form within or on the device and to methods of preparing and using such devices. The temporal profile of release of the bioactive from the device in an aqueous medium is determined by the relative amounts of the non-ionic and ionic forms.

### BACKGROUND

Medical devices have been combined with antibiotics for more than 60 years. In U.S. Pat. No. 2,237,218, Flynn teaches that an antiseptic, local anesthetic, fungicide, bactericide, medicament or other addition of therapeutic value may be incorporated into a catheter by dissolving the same in a solvent and impregnating it into a cellulose substrate. The solvent is preferably removed by an evaporative step so that the medicament is uniformly distributed throughout the substrate.

U.S. Pat. Nos. 2,976,576 and 3,220,960 teach that hydrophilic hydrogel materials have the ability of absorbing large quantities of therapeutic solutions. In U.S. Pat. No. 3,428,043, Shepherd subjects thin flexible sheets of hydrogel to a therapeutic solution for time sufficient for the solution to be absorbed. Heating and/or drying followed and the sheet was stored in its anhydrous state.

In U.S. Pat. No. 4,605,564, a medical implant device is made antimicrobial by treating the substrate with para-chloro-meta-xylenol dissolved in an organic solvent, such as dichloromethane or carbon tetrachloride, to cause swelling of the polymer. The solvent is then removed to trap or precipitate the drug in the polymer.

In U.S. Pat. No. 4,612,337, Fox prepares an infection-resistant polymeric material using soaking steps. A substrate was soaked with an antimicrobial agent dissolved in an organic solvent. The substrate is then soaked with an organic solvent with a metal salt incorporated. Subsequently, the substrate is soaked with the antimicrobial agent dissolved in the organic solvent. The substrate is dried after each soaking step. This method is said to create long lasting antimicrobial effects and biphasic release of the antimicrobial agents. The amount of antimicrobial agent incorporated into the device is said to be appreciably increased with the inclusion of the intermediate soaking step.

In U.S. Pat. No. 4,865,870, Hu renders a substrate antithrombogenic by first incorporating a surface active agent, such as dodecylmethylamine, into the substrate. The pH of the coating solution is adjusted with an alkalinizing agent, such as a water soluble amine, ammonium hydroxide or an alkali metal hydroxide. The substrate is then reacted with a heparin solution so that the heparin reacts with the surface active agent and is incorporated into the substrate. The quantity and duration of heparin bonding to the substrate is said to be increased compared to prior art heparinization procedures. The chelation of the negatively charged heparin molecule to the surface active agent causes the increased duration of bonding of the heparin drug.

In U.S. Pat. No. 5,069,899, Whitbourne reacts heparin with a quaternary ammonia component and a water-insoluble polymer. Remaining quaternary ammonia components are reacted with negatively charged antibiotics. This process is said to result in a more gradual release of the drugs over time. No manipulation of the elution profile is discussed.

In U.S. Pat. No. 4,917,686, Bayston prepares a medical device antimicrobial by using a swelling agent containing a dissolved antimicrobial agent. A polymeric material is contacted by the swelling agent for a sufficient period of time to promote swelling of the substrate thereby causing diffusion and migration of the antimicrobial agent into the enlarged intermolecular spaces of the substrate. The solvent is then removed so that the intermolecular spaces return to their original size and shape with the antimicrobial agent uniformly distributed for subsequent continuous migration to and diffusion through the surfaces. No manipulation of the elution profile is discussed.

In U.S. Pat. Nos. 5,624,704 and 5,902,283, Darouiche impregnates a non-metallic medical implant with an antimicrobial agent. This method includes dissolving an effective amount of antimicrobial agent in an organic solvent, and adding a penetrating agent and an alkalinizing agent to the composition under conditions in which the antimicrobial composition permeates the material of the medical implant. The alkalinizing agent, such as sodium hydroxide, enhances the reactivity of the substrate. The penetrating agent, such as ethyl acetate, promotes penetration of the antimicrobial agent into the material of the medical device. This method of impregnation results in long efficacy profiles due to a greater amount of antimicrobial substance being imparted to the substrate. No manipulation of the elution is discussed.

In U.S. Pat. No. 6,716,895, Terry describes the tailoring of elution profiles of various metal salts (e.g. silver nitrate, silver chloride, etc.) from polymers.

### SUMMARY

One aspect provides a medical device including a substrate having at least one surface and a bioactive in a salt and a free-base form in contact with the substrate. The salt and free-base form of the bioactive have different solubilities in an aqueous medium. The free-base form may include an amine.

In one embodiment at least 98% of the bioactive is present in the free-base form less than 2% of the bioactive is present in the salt form. In another embodiment, at least 98% of the bioactive is present in the salt form and less than 2% of the bioactive is present in the free-base form.

In various embodiments, the medical device is a catheter, drain tube, external fixation pin, orthopedic plate, endotracheal tube, stent, shunt, feeding tube or a hernia mesh.

In other embodiments, the substrate is a non-metallic substrate comprising a polymer selected from the group consisting of elastomers, natural or synthetic rubbers, silicones, polyurethanes, polyisoprene, polycarbonates, plastics, polyethylenes, polypropylenes, polyvinyl chlorides, polyvinyl alcohol, polyvinylpyrrolidones, polyvinyl acetate, polyetherketones, polyamides, polyvinylidene chloride, polystyrene, polyacrylates, methacrylates, polyesters, poly(ethylene glycol) and poly(lactic acid-co-glycolic acid) and combinations thereof.

In various embodiments, the bioactive is an antimicrobial, antiseptic, anesthetic or an anticoagulant. In certain embodiments, the antimicrobial is a lincosamide, clindamycin, a aminoglycoside, gentamicin, a tetracycline, minocycline, a, fluoroquinolone, ciprofloxacin, a polypeptide, polymyxin, a glycopeptide, vancomycin, an aminocyclitol, spectinomycin, a macrolide, erythromycin, rifampin, a glycylcycline, tigecycline, a sulfonamide, or sulfadiazine. In other embodiments, the anesthetic is bupivacaine, ropivacaine, lidocaine, chloroprocaine, pramoxine or mepivacaine. In yet another embodiment, the anticoagulant is heparin.

In another embodiment the medical device includes a second bioactive in a salt and a free-base form in contact with the substrate. The salt and free-base form of the second bioactive have different solubilities in an aqueous medium. In certain embodiments the bioactive and the second bioactive are antimicrobials. In other embodiments, the bioactive is an antimicrobial and the second bioactive is an antiseptic. In yet other embodiments, the bioactive is an antimicrobial and the second bioactive is an anesthetic.

In another embodiment, the substrate is a nonmetallic substrate and the bioactive is contained within the nonmetallic substrate.

In yet another embodiment, the substrate is a metal, metal alloy, glass, ceramic or a mineral.

Another aspect provides a method for manufacturing a medical device. The method includes contacting a solvent with a bioactive in a salt form and a free-base form to form a solution. The salt form is more soluble in an aqueous medium than is the free-base form. The method also includes contacting a surface of the medical device with the solution as that the salt and free-base form are impregnated into the medical device or are coated onto the surface of the medical device.

Another aspect provides a method for controlling the elution profile of a bioactive. The method includes contacting a solvent with a bioactive in a salt form and a free-base form and manipulating the polarity of said solvent to adjust the ratio of the free-base to salt forms of the bioactive.

Yet another aspect provides a medical device comprising: substrate having at least one surface; and a bioactive and a salt of the bioactive in contact with the substrate, wherein the bioactive and its salt have different solubility and a different dissolution rate in a aqueous medium. The bioactive is an organic base, an organic acid or a zwitterionic compound.

Another aspect provides a method for manufacturing a medical device. The method includes contacting a substrate having at least one surface with a solution comprising a solvent, a bioactive and a salt of the bioactive. The bioactive and its salt are impregnated into the medical device or coated onto the surface of the medical device in a ratio dependent on the polarity or dielectric constant of the solvent. The bioactive is an organic base, an organic acid or a zwitterionic compound.

### BRIEF DESCRIPTION OF THE DRAWINGS

FIG. 1 is an equilibrium diagram of two precipitation pathways for a free-base (represented by capitalized B) and free-base-salt complex (B—HCl). The equilibrium between the protonated form of a free-base (B—H⁺) and the free-base (B) is described in an aqueous solution as the pkₐ of B—H⁺.

FIG. 2 is an equilibrium diagram of two precipitation pathways for a free-base and free-base-salt complex. The influence of two solvents is illustrated in Precipitation routes 1 and 2. Precipitation route 1 depicts the influence of a polar solvent such as water on the equilibrium. Precipitation route 2 depicts the influence of a less polar solvent such as ethanol on the equilibrium. Thickness of arrows denotes preferential direction of equilibrium. However, when just the free-base form of bupivacaine is used in ethanol only the free-base form is deposited/impregnated.

FIG. 3 is a graph showing the total load for bupivacaine-HCl imbibed stents as a function of daily elution (over 2 days). The stents were imbibed in a 5% bupivacaine solution at room temperature for 15 minutes. The solvents used were 95% ethanol and nanopure water.

FIG. 4 is a graph showing the total load for bupivacaine-HCl imbibed stents as a function of solvation polarity.

FIG. 5 is a graph showing day 1 elution for bupivacaine-HCl imbibed stents as a function of solvation polarity.

FIG. 6 is a graph showing day 2 elution for bupivacaine-HCl imbibed stents as a function of solvation polarity.

### DESCRIPTION OF THE PREFERRED EMBODIMENTS

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety.

The materials, methods, and examples disclosed herein are illustrative only and not intended to be limiting. In particular, those skilled in the art will recognize that there are a number of different methods of combining bioactives(s) with a medical device. These include incorporation of bioactive(s) within the substrate material, on the substrate surface(s), and within a coating layer(s) on the surface(s) or any combination of these methods. All such methods are within the scope of the embodiments described herein.

Medical Devices Including Non-ionic and Ionic Forms of a Bioactive

One aspect of the present invention provides a medical device containing an ionic form and a non-ionic form of a bioactive, where the ionic and non-ionic forms have differing solubilities in an aqueous medium. For example, the device can contain a bioactive having a site available for protonation and a salt formed by protonation of this site.

Because the ionic and non-ionic forms have differing solubilities, the elution of the bioactive from such devices in an aqueous medium differs from the elution profile of the bioactive from a device containing only the ionic or the non-ionic form. The elution rate in an aqueous medium is determined by the ratio of the ionic form to the non-ionic form.

In one embodiment, at least 98% of the bioactive is present in the non-ionic (free-base or free-acid) form and less than 2% of the bioactive is present in the ionic (salt) form. In another embodiment, at least 98% of the bioactive is present in the ionic (salt) form and less than 2% of the bioactive is present in the non-ionic (free-base or free-acid) form. In other embodiments, at least 75%, 50% or 25% of the bioactive is present in the non-ionic (free-base or free-acid) form and less than 25%, 50% or 75% of the bioactive is present in the ionic (salt) form.

In certain embodiments, the ionic and non-ionic forms are contained in a surface coating present on the surface of the device. In other embodiments, the non-ionic and ionic forms are contained within the device. For example, these forms have be impregnated or imbibed into a polymeric material that forms at least part of the medical device. In still another embodiment, the bioactive is both contained within the device and is present on the surface of the device and/or on the surface in a coating material.

In one embodiment, a bioactive is incorporated within or on a medical device by making a concentrated to super-concentrated solution of the bioactive and depositing the bioactive onto the surface or into pores present in the device. Such pores may be part of the structure of a non-metallic substrate of the device. For example, such a substrate may include silicone, polyurethane or polyvinyl alcohol. Pores may also be created by forming a mineral layer on the surface of a substrate. In another embodiment, bioactive incorporation is achieved by swelling a polymeric substrate with a suitable swelling agent to create a larger and more complex series of pores and incorporating the bioactive within such pores.

In various non limiting embodiments, the medical device is a catheter, drain tube, external fixation pin, orthopedic plate, endotracheal tube, stent, shunt, feeding tube, or hernia mesh. The medical device can be made from a non-metallic material or a metallic material with a mineral coating and/or non-metallic coating. The medical device, or surface coating of the medical device, may be made from a polymer or combination of polymers including copolymer(s). Some examples are, but not limited to, elastomers, natural rubbers, synthetic rubbers, silicones, polyurethanes, polyisoprene, polycarbonates, plastics, polyethylenes, polypropylenes, polyvinyl chlorides, polyvinyl alcohol, polyvinylpyrrolidones, polyvinyl acetate, polyetherketones, polyamides, polyvinylidene chloride, polystryene, polyacrylates, methacrylates, polyesters, poly(ethylene glycol), poly(lactic acid-co-glycolic acid) and copolymers and combinations thereof. The medical device may also be an injectable sol-gel or similar delivery device. In yet other embodiments, the medical device includes a metal, metal alloy, glass, ceramic or a mineral.

The bioactive can be any bioactive compound or combination of bioactive compounds that exist in an ionic and non-ionic form as described above. Some non limiting examples of bioactives are antimicrobials, antiseptics, anesthetics, and anticoagulants. Certain embodiments provide a device useful for the prevention and/or control of bacterial colonization on the implanted device through incorporation of an antimicrobial, antiseptic and/or anticoagulant or combination thereof. Certain other embodiments provide a device incorporating a bioactive agent useful for the reduction of pain associated with the physical presence of the device in the body. In other embodiments, the device includes both a bioactive that controls pain and a bioactive that and controls bacterial colonization.

Examples of antimicrobials include lincosamides (e.g. clindamycin), aminoglycosides (e.g. gentamicin), tetracyclines (e.g. minocycline), fluoroquinolones (e.g. ciprofloxacin), polypeptides (e.g. polymyxin), glycopeptide (e.g. vancomycin), aminocyclitols (e.g. spectinomycin), macrolides (e.g. erythromycin), rifampin, glycylcyclines (e.g. tigecycline) and sulfonamides (e.g. sulfadiazine).

Examples of antiseptics include chlorhexidine and benzalkonium. Examples of anesthetics include to bupivacaine, ropivacaine, lidocaine, chloroprocaine, pramoxine and mepivacaine. Examples of anticoagulants include heparin and its salts.

In certain embodiments, the medical device includes the non-ionic form of one bioactive and the ionic form of a second bioactive.

In other embodiments, the device includes the ionic and non-ionic forms for a first and a second bioactive. In one embodiment, the first and the second bioactives are antimicrobals. For example, the first bioactive can be clindamycin and the second bioactive can be minocycline. In another embodiment, the first bioactive is an antibiotic and the second bioactive is an antiseptic. In yet another embodiment, first bioactive is an antimicrobal and the second bioactive is an antiseptic. In another embodiment, first bioactive is an antimicrobal and the second bioactive is an anesthetic. In other embodiments, the medical device may also include antimicrobial metals such as silver metal and silver salts.

Bioactive Compounds Having Non-Ionic and Ionic Forms

Certain bioactives (B) include one or more sites that are available to be protonated or deprotonated to form a salt of the compound. In such an embodiment, the bioactive can be a free-acid, a free-base or a “zwitterionic” form having free-base and free-acid components. For the purposes of the present invention, the salt form is considered to exist as the protonated free-base (B—H⁺) or deprotonated free-acid (A⁻) and an appropriate counter ion. The unmodified compound (B) is referred to as the non-ionized form of the compound while the salt of the compound is referred to as the ionized form.

In solution, the ionic form and non-ionic forms exist in equilibrium. The ratio of these forms is determined by factors such as the pH, drug pKₐ, temperature, and polarity of the solution (if the solvent is an organic solvent or mixture of water and a less polar solvent). Thus, for a base drug-salt including an Cl⁻ counter ion, the ionic form B—HCl (B—H⁺ (ionized)+Cl⁻) exists in equilibrium with the non-ionic form B. For an acid drug-salt having a Na⁺ counter ion, the ionic form A-Na (A⁻(ionized)+Na⁺) exists in equilibrium with the non-ionic form (AH).

The formation of the ionic form requires that the non-ionic form of the bioactive includes a site available for protonation or deprotonation. On course, certain bioactives can contain both protonation or deprotonation sites. For the purposes of the present invention, the terms ionic and non-ionic have used to describe the state of protonation or deprotonation of a site on the bioactive.

In the devices described herein, the protonated and deprotonated forms of the bioactive have differing solubilities in an aqueous medium. The ratio of protonated to deprotonated bioactive present in and/or on the device is chosen to achieve a required elution profile in such a medium. In one embodiment, the protonation site is a quaternary amine structure. One example of a bioactive having such a site is the anesthetic bupivacaine. Bupivacaine may be converted to an ionic form by adding the non-ionic form of the drug to an excess of concentrated hydrochloric acid in solution. The solution is then dried by evaporation forming the ionic form bupivacaine-HCl.

Some examples of counter ions present in the ionic form of protonated (B—H⁺) bioactives include chloride, glucuronate, sulfate, benzoate, bicarbonate, citrate, estolate, iodide, lactate, maleate, nitrate, pamoate, succinate, tartrate, diflunisal and other aromatic hydroxycarboxylic acids. Many other examples, including some salts of deprotonated weak acids are described in Gould P L, Salt selection for basic drugs, Int J Pharma 33:201-217 (1986).

Methods of Forming Devices Having Non-ionic and Ionic Forms of a Bioactive

Another aspect provides methods for treating a substrate of an implantable medical device to achieve a required ratio of the ionic to non-ionic forms. The methods provide for tailoring the amount and temporal profile of elution of the bioactive from the medical device by incorporating a controlled ratio of non-ionic and ionic forms of the bioactive. In one embodiment, the non-ionic and ionic forms are incorporated into or onto the device through contact of the device substrate with a solution of solvent(s) containing the ionic and non-ionic forms.

Certain embodiments provide a method for altering the ratio of the ionic and non-ionic forms of bioactive that are incorporated with the device by the selection of the solvent(s) based on polarity, dielectric constant, and intrinsic ability to donate or accept protons. If a solvent has a greater capacity to solvate protons than another solvent, it is considered to be more basic relative to the other solvent. This is advantageous if the desired effect is to deprotonate a protonated free-base (B—H⁺ becomes B) and to increase deposition of the non-ionic form. Similarly, such a solvent can deprotonate a neutral free-acid (HA becomes A⁻) and increase the deposition of the ionic form. Hence, the-selection of solvent(s) based on polarity, dielectric constant and a solvents capacity to donate or solvate protons is of great importance for the method of incorporating a controlled fraction of the ionic and non-ionic forms on a medical device. The properties of polymers used in a coating composition applied to the surface of a medical device can also impact the ratio of ionic and non-ionic forms of the bioactive deposited on the device. Another embodiment provides a method by which the ratio of ionic and non-ionic forms is directly manipulated by mixing known amounts of non-ionic and ionic forms.

Certain embodiments provide a method of preparing a medical device including a required ratio of the ionic and non-ionic forms of a bioactive, for example 90% non-ionic form and 10% ionic form. Such embodiments use a solvent system that solubilizes both the ionic and non-ionic forms at a required pH. The use of organic solvents is often essential for incorporating sizeable amounts of bioactive on the medical device. In certain embodiments, the organic solvent may be selected from the group consisting of alcohols, ketones, aldehydes, ethers, chloroform and methylene chloride. The solubility of the ionic form of the bioactive must also be taken into account. Here, the use of organic salts is effective for increasing solubility.

As organic solvent concentration increases, the pKₐ of protonated organic bases decreases and for organic acids the pkₐ increases, relative to the pkₐ in aqueous solution. Temperature can also have a significant effect on the pkₐ of weak acid or base bioactives. The right combination of polar and non-polar solvents at the appropriate pH, salts, ionic strength, and temperature, for a given pkₐ, is necessary for producing a medical device with sufficient quantity of bioactive and with the proper non-ionic to ionic ratio for the designed elution rates. In controlling the quantities and ratio of non-ionic to ionic deposition, within and on a nonmetallic substrate, it is necessary to use a solvent system that optimizes the ratio and solubility of both the non-ionic and ionic forms. In certain embodiments, the pH and pKₐ(s) of the imbibing and/or coating solution are adjusted by selection of temperature and solvent composition; and/or the quantities and relative ratios of a non-ionic (free-base or free-acid) forms and their salts. For example, a solution's pH can be increased by the addition of a free-base or free-acid-salt, both of which can be proton acceptors, or a combination thereof. Additionally, the pH can be decreased by the addition of a free-base-salt or free-acid both of which can be proton donors, or a combination thereof. The solvent composition can include solvents selected from, but not limited to, water, aromatic hydrocarbons (e.g., xylene), chlorinated hydrocarbons (e.g., chloroform), esters/acetates (e.g., ethyl acetate), aliphatic hydrocarbons (e.g., hexane), cycloalkanes (e.g., cyclohexane), alcohols (e.g., hexanol), nitriles (e.g., acetonitrile), ketones (e.g., acetone), amines (e.g., triethanolamine), heterocyclic solvents (e.g., tetrahydrofuran), ethers (e.g., diethyl ether), heterocyclic solvents (e.g., tetrahydrofuran), aldehydes, methylene chloride and any combination thereof.

The selection of an organic solvent or mixture of solvents for their intrinsic ability to donate or accept protons is very important for producing the necessary combination of ionized and unionized forms of bioactive(s) in solution and for controlling the ratio of bioactive and bioactive-salt deposition.

The mixtures of ethanol and water are termed co-solvents. Some common co-solvents used in pharmaceutics are, but not limited to, polyethylene glycol, ethanol, methanol, sorbitol, and propylene glycol. Certain embodiments include the use of a polar alcohol, instead of water for solubilizing salts and the ionized form of bioactives, in combination with an organic solvent. Examples include methanol and ethanol. The use of co-solvents may increase the solubility of different bioactive forms appreciably. This is due to the reduction of the dielectric constant of the solvent. Again, the reduction of the dielectric constant has repercussions on the cleaving of the free-base-salt complex as well.

Certain embodiments provide methods for controlling the relative deposition of a bioactive and its salt within or on porous medical devices with or without additional coatings. The pores can be an intrinsic and/or manufactured property of a polymer or created by swelling a non-metallic substrate with a swelling agent at an appropriate temperature. In other embodiments, a mineral layer is formed on a metallic or non-metallic substrate. In yet other embodiments, by a polymer coating layer containing the ionic and non-ionic forms is formed on a metallic or non-metallic substrate.

In certain embodiments, the rate of deposition between the non-ionic and ionic forms of the bioactive on and within the material of the medical device differs. Because of this, the ratio of the non-ionic and ionic forms of bioactive in the coating or imbibing solution will not necessarily equal the ratio of non-ionic and ionic forms on the device. The hydrophobic/hydrophilic properties of the substrate material are an important factor in the bioactive deposition ratio and the elution rates. Hence, the hydrophobic/hydrophilic properties of the substrate, the non-ionic and ionic forms of the bioactive all contribute to the bioactive elution rate.

Differential Deposition of a Bioactive Free-Base and its Salt by Means of Selection of the Polarity of the Solvent

Dissolution of a solute in a polar solvent, such as water, involves at least two steps. First, the dipoles of the solvent interact with the dipoles of the solute. If the solvent dipole is of sufficient magnitude, this interaction results in the breakage of the ionic bond between the free-base and the salt. Second, entropy causes dispersion of the solute in the solvent. The dipole/dielectric constant for water:methanol:ethanol are 1.85/80:1.70/33:1.69/24.3 respectively. Both methanol and ethanol are less polar than water. Ethanol has a lower dielectric constant and therefore a lower solubility equilibrium constant (Kₛₚ) for inorganic salts. For example, the number of grams of NaCl soluble in 100 grams of solvent is 35.92 grams for water and 0.065 grams for ethanol.

Thus, the salient feature governing the extent that a solute dissolves in a specified solvent is the interaction of their respective dipoles. If a free-base-HCl complex is solved by a polar solvent such as water, the complex is cleaved first with the removal of Cl⁻ and then, depending on pH conditions, by the removal of the H⁺. On the other hand, if a free-base-HCl is solved by a much less polar solvent, the dipoles are not available to cleave the Cl⁻ and the H⁺. Thus, for such less polar solvents, the free-base-salt can remain intact while in solution. In such cases, the free-base-salt is solved by induced dipoles and hydrogen bonds.

This solvent-solute interaction is illustrated by the extremes. An ideal solution has no attractive or repulsive forces between the solvent and the solute. The ideal solvent is completely nonpolar and does not result in the breakage of the ionic bond between a free-base and salt. Nonideal solutions are those with interactions between solvent and solute.

By choosing solvents with respect to their dipole and dielectric constant, the relative amount of solvation and subsequent deposition onto a medical device of free-base-salt (ionic form) to the free-base (non-ionic form) may be controlled. For example, in the case of bupivacaine-HCl, more polar solvents will tend to cleave the bond between the free-base-H⁺ (bupivacaine-H⁺) and Cl⁻ to form a solution based on the attractive forces of dipole interactions. Less polar solvents will tend to hold the free-base-salt complexes in solution by London forces and hydrogen bonds. Thus, the choice of solvent will determine the ratio of free-base-salt complex (ionic form) to free-base (non-ionic form) eventually contained on a medical device.

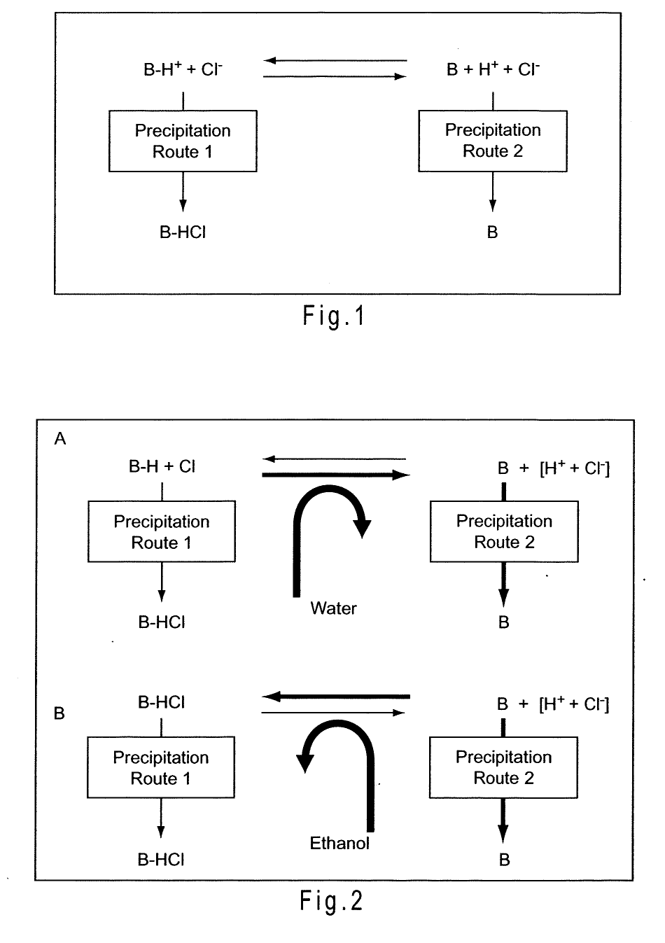

FIG. 1 illustrates two precipitation pathways for a free-base (B) and its salt (B—HCl). The first precipitation pathway involves the salt and is designated “Precipitation route 1” in FIG. 1. The second precipitation pathway involves the dissociated free-base and is designated “Precipitation route 2” in FIG. 1. The relative amounts of solute precipitated in a solvent by either of these routes is determined by the quantity and relative ratio of free-base and salt in solution (i.e., the pH, temperature, and pKₐ of the free-base).

In solution, the relationship between the percentage of the free-base and salt forms is given by:

$${pk}_{a} = - log \frac{[ B ] [ {H}^{+} ]}{[ B - {H}^{+} ]}$$

Here [B] is the concentration of the free-base (non-ionic form) and [B—H⁺] is the concentration of protonated free-base (ionic form).

The relative importance of the two precipitation pathways differs for different solvents. The relative amounts of solute deposited by either of these routes onto and/or into a non-metallic substrate can be determined for a given set of conditions such as pKₐ, pH and temperature, and the chemical properties of bioactive(s), substrate(s), solvent(s), and salt(s) used. When the pH is equal to the pKₐ, the ratio of free-base to salt is one. However, the pKₐ doesn't determine the solubility of the ionized or unionized forms in a given solvent system.

This relationship may be illustrated using the specific case of bupivacaine-HCl. Less energy is required for bupivacaine-HCl to form ions in water than in ethanol. The energy of separation is inversely proportional to the dielectric constant, which is 0.0123 for water and 0.0412 for ethanol. It takes less energy to keep the bupivacaine-H⁺ and Cl⁻ ions separated in water than in a less polar solvent. Hence, the bupivacaine-salt (B—HCl) is less likely to deposit on a device surface at a given concentration in an aqueous solution than in a less polar solvent. Deposition of the salt form of bupivacaine-HCl more readily occurs in ethanol based solutions for this reason.

FIG. 2 is an equilibrium diagram of two precipitation pathways for a free-base and salt showing the relative importance of Precipitation routes 1 and 2 for water (polar solvent) and ethanol (non-polar solvent). Thickness of arrows denotes preferential direction of equilibrium. Presence of a more polar solvent favors precipitation by route 2, i.e. the deposition of the free-base form. Presence of a less polar solvent favors precipitation by route 1, i.e. the deposition of the salt form. If mixtures containing different ratios of a polar solvent (for example, water) and a less polar solvent (for example, ethanol) are used, such as 25% ethanol to 75% water, 50% ethanol to 50% water, or 75% ethanol to 25% water, the ratio of salt to the free-base deposited onto a device may be controlled.

For those compounds for which the free-base and salt forms have different solubilities in an aqueous medium, controlling the ratio of free-base to salt forms on the device allows for control over the elution profile of the device. For example, free-base bupivacaine is at least two orders of magnitude less soluble in aqueous solution, than the bupivacaine-salt. Consequently, the rate of elution of the free-base bupivacaine in an aqueous medium will be less than the rate of elution of the bupivacaine-salt. Thus, the elution profile for bupivacaine in the aqueous medium of the body may be tailored by controlling the quantities of the free-base and the salt forms present on the device by selecting an appropriate solvent.

The mixtures of ethanol and water are termed co-solvents. Some examples of common co-solvents used in pharmaceutics include polyethylene glycol, ethanol, methanol, sorbitol, and propylene glycol. In certain embodiments, a polar alcohol can be used instead of water in combination with an organic solvent. Examples include methanol and ethanol. The use of co-solvents may increase the solubility appreciably. This is due to the reduction of the dielectric constant of the solvent. Again, the reduction of the dielectric constant has repercussions on the cleaving of the free-base-salt complex as well. In controlling the quantities and ratio of bupivacaine to bupivacaine-salt deposition, within and on a non-metallic substrate, it is necessary to use a solvent system that optimizes the ratio and solubility of both the free-base and the protonated free-base (B—H⁺) and a salt (both organic and inorganic) of a basic drug.

In certain embodiments, bupivacaine-HCl is deposited onto a polyurethane stent. A required temporal elution profile in an aqueous medium is obtained by manipulating the polarity of the solvent to adjust the ratio of bupivacaine (free-base) to bupivacaine salt. In one such embodiment, the bupivacaine is deposited in a calcium phosphate layer on the surface of a polyurethane stent. The calcium phosphate is a porous coating that erodes with a zero order rate coefficient. The bupivacaine is put into the pores of the coating using a process of imbibing, consisting of the controlled deposition of bupivacaine onto the stent when immersed in a solvent.

In other embodiments, the precipitation rate is increased by cooling the solvent environment around the stent, for example, by freezing the stent prior to immersion in the imbibing solution. In other embodiments, the solubility of an acidic or weakly basic bioactive is perturbed by transiently varying the pH to cause increased precipitation of the bioactive. The deposition rate and quantities of bioactive deposited may also be altered by controlling the free-base and salt concentrations, as well as the solvent composition.

In water, the free-base (unionized form) of bupivacaine is much less soluble in water than the salt form (bupivacaine-HCl). A water suspension of free-base in water has a pH of 7.85. At this pH, the solubility of bupivacaine is about 0.25 mg/ml. By lowering the pH, the ratio of protonated bupivacaine (ionized) to unprotonated bupivacaine (unionized) increases. The ionized form is significantly more soluble in water with 40-50 mg/ml dissolved with a pH between 1.8 and 6.

The differential solubility of bupivacaine relative to bupivacaine-HCl has an impact on the elution profile from a coated ureteral stent placed in a urine stream. The free-base form of bupivacaine is poorly soluble in an aqueous medium and elutes slower than the salt form. Thus, controlling the ratio of free-base and the bupivacaine salt imbibed on the stent allows for proper rates of drug release.

A more complete understanding of the present invention can be obtained by reference to the following specific examples. The Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitations. Modifications and variations of the invention as herein before set forth can be made without departing from the spirit and scope thereof, and, therefore, only such limitations should be imposed as are indicated by the appended claims.

### Example 1

### Mineralization of a Polyurethane Ureteral Stent

The surface of the polyurethane ureteral stent (Cook Group Inc.) is first prepared using cold gas plasma to enhance adhesion of a calcium phosphate coating. The plasma process consists of a vacuum step to allow for out-gassing of the polymer. This is followed by oxygen cleaning and silane ((3-Glycidoxypmpyl)trimethoxysilane) functionalization. Silane is introduced into the plasma chamber by a flash evaporator.

The stents are prepared for calcium phosphate mineralization as described by Campbell (U.S. Pat. No. 5,958,430) and Tarasevich (U.S. Pat. No. 5,759,708), the contents of which are incorporated herein by reference. The plasma functionalized stents are treated with sodium sulfite solution by soaking in 0.1 M Na₂SO₃ for 15 hours at 20° C. The stents are then rinsed in water and dried before being submerged in 0.1 N HCl for 5 minutes at 20° C. After again being rinsed and dried, a calcium phosphate mineralization layer is applied by immersing the stents in a solution containing 5 mM CaCl₂, 1.5 mM KH₂PO₄, and 1.5 mM Na₂HPO₄ for 1 hour at 30° C. The coated stents are again rinsed and dried.

### Example 2

### Effect of Solvent Polarity on Deposition of Bupivacaine

The effect of polarity of coating solvent on the deposition of bupivacaine-HCl and bupivacaine on calcium phosphate coated polyurethane stents is demonstrated by imbibing the stents using solvents containing different ratios of ethanol and water. Bupivacaine-HCl solubility in water and ethanol are 40 g/liter and 125 g/liter respectively. Stents, prepared as described in Example 1, are imbibed in 5% bupivacaine-HCl in water/ethanol for 20 minutes at 45° C.

The following percentages of ethanol-water solutions: 0%-100%; 25%-75%; 50%-50%; 75%-25%; and 100%-0% were used. The stents are then blown dry at 60° C.

The amount of drug deposited on the stent is determined by high performance liquid chromatography (HPLC). The rate of bupivacaine dissolution is determined by measuring the elution profiles using HPLC.

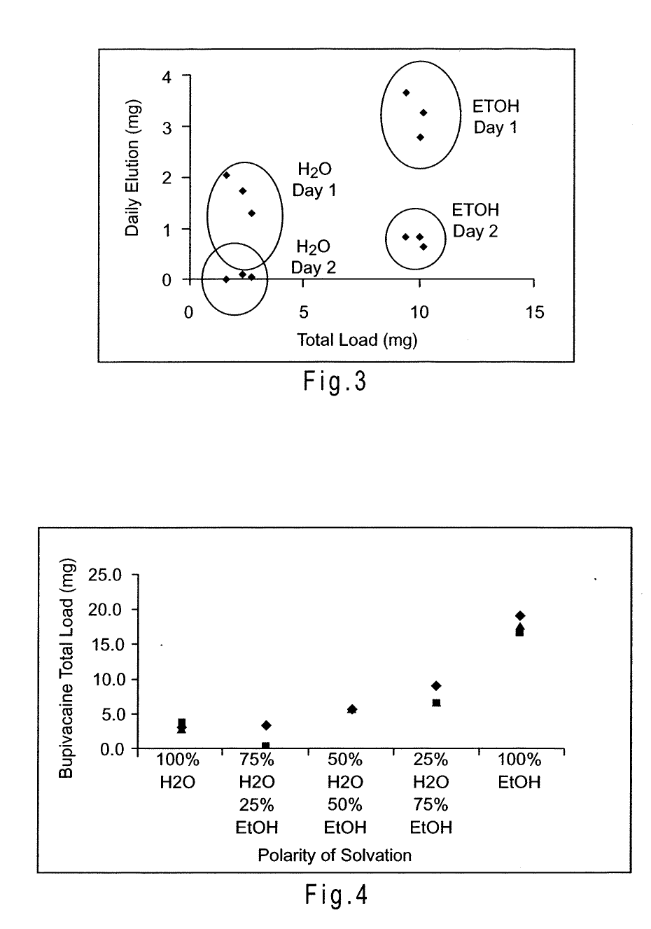

If the polyurethane stent is imbibed with bupivacaine free-base, the rate of elution of drug is greatly reduced, however, if imbibed with bupivacaine-HCl the drug can elute more quickly. Ethanol bupivacaine-HCl (5% in bupivacaine-HCl) solutions deposit more bupivacaine on calcium phosphate coated polyurethane stents with a larger ratio of bupivacaine-HCl to free-base than water bupivacaine-HCl (5%) solutions (see FIG. 3).

In a comparison of ethanol and water solvents for imbibing bupivacaine onto polyurethane stents, it was found that ethanol resulted in a total bupivacaine load of 10 mg and water produced a total bupivacaine load of 3 mg. This measure does not define the solubility of the deposited drug. The solubility and thus the ratio of bupivacaine to bupivacaine-HCl was determined by the elution of the deposited drug in buffered saline solution (pH=7.4) mimicking a bodily fluid. The elution of the drug on day one and day two for the ethanol solution were approximately six times that of the water solution. Thus, the solubility of the deposited drug was close to six times greater for ethanol deposition than for water deposition.

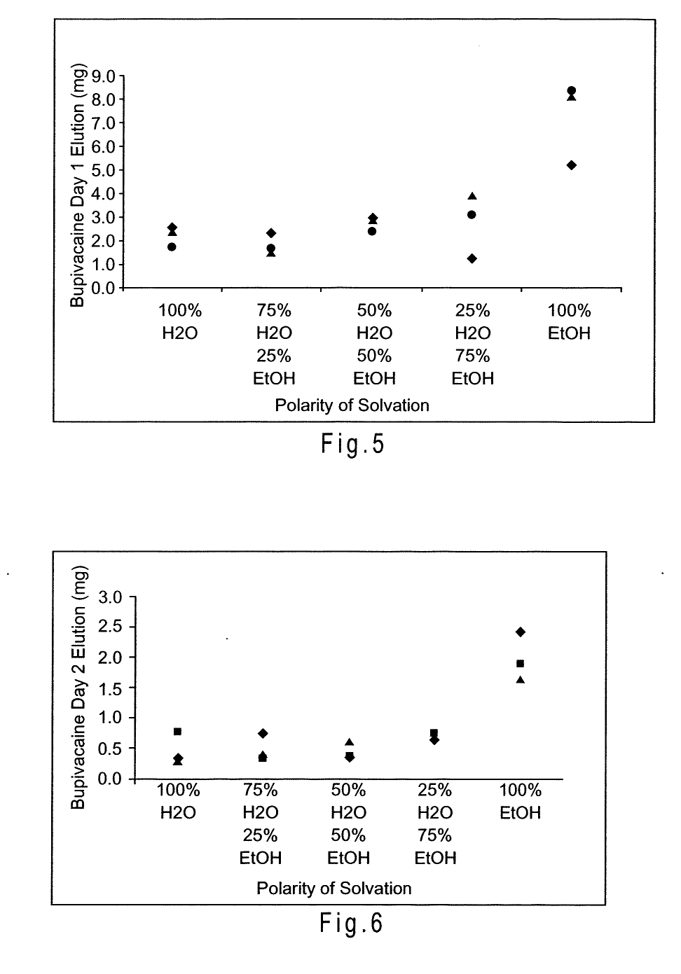

Another series or experiments were conducted using a five solvent solutions, ranging from 100% water (highly polar) to 100% ethanol (less polar). To each solvent, 5% bupivacaine was added. Stents were imbibed at 45° C. for 20 minutes. Total load (FIG. 4), day 1 elutions (FIG. 5), and day 2 elutions (FIG. 6) were measured.

### Example 3

### Tailoring the Temporal Elution Profile by Directly Manipulating the Ratio of Bupivacaine Free-Base to Bupivacaine Salt

The ratio of free-base to free-base-salt can be directly manipulated by combining known amounts of free-base and free-base-salt. Medical grade polyurethane stents as prepared above was placed in an ethanol solutions containing various ratios of bupivacaine-HCl to bupivacaine (see Table 1) at a concentration of 10% by weight. In this case the polyurethane stents did not contain a calcium phosphate layer.

TABLE 1
Ethanol charged with various ratios of free-base-salt
to free-base; totaling 10% bupivacaine by weight

| | | Bupivacaine free-base |
|---|---|---|
| Experiment | Bupivacaine-HCl(%) | % |
| Series 1 | 75 | 25 |
| Series 2 | 50 | 50 |
| Series 3 | 25 | 75 |
| Series 4 | 12.5 | 87.5 |
| Series 5 | 0 | 100 |

The ethanol solution was heated to 45° C. for 1 hour. In this process, the ethanol was used as a swelling and penetrating agent, as well as, a solvent. The substrate was taken out of solution then allowed to dry.

TABLE 2
Elution from Stent on Day One and Day Two

| | Day 1 Elution | Day 2 Elution | Remaining Load |
|---|---|---|---|
| | (mg) | (mg) | (mg) |
| Series 1 | 5 | 1.8 | 3.2 |
| Series 1 | 7.1 | 1.5 | 3.4 |
| Series 2 | 4.7 | 2.9 | 4.7 |
| Series 2 | 4.9 | 4 | 6.3 |
| Series 3 | 5.4 | 3 | 6.5 |
| Series 3 | 5.4 | 3.9 | 6.8 |
| Series 4 | 6.4 | 5.6 | 2.5 |
| Series 4 | 5.9 | 6.4 | 13.2 |
| Series 5 | 4.8 | 5.1 | 9.2 |
| Series 5 | 4.8 | 4.1 | 7.3 |

Table 2 shows the day one and day two elution amounts of bupivacaine, along with the remaining total bupivacaine load after the two-day elution. As the free-base fraction is increased (progression from Series 1 to 5), the day two to day one bupivacaine ratios are increased; as well as, the remaining bupivacaine (extracted using HCl and acetonitrile) to day two ratios. Thus, as the fraction of free-base is increased the total amount of drug that is deposited, within and on the polyurethane material, increases and the day two bupivacaine relative to day one increases. These results indicate that a larger quantity of the more slowly eluting free-base form of bupivacaine is deposited on the stent. The tailoring of the elution profile by the manipulation of the fraction of free-base deposited is most pronounced after day one, with only a moderate effect evident on day one.

We claim:

1. A medical device comprising:
a substrate having at least one surface; and
a first bioactive in a salt and a free-base form in contact with the substrate,
wherein the salt and free-base form of the first bioactive have different solubilities in an aqueous medium.

2. The medical device of claim 1, wherein free-base form comprises an amine.

3. The medical device of claim 1, wherein at least 98% of the first bioactive is present in the free-base form less than 2% of the first bioactive is present in the salt form.

4. The medical device of claim 1, wherein at least 98% of the first bioactive is present in the salt form and less than 2% of the first bioactive is present in the free-base form.

5. The medical device of claim 1, wherein the medical device is selected from the group consisting of a catheter, drain tube, external fixation pin, orthopedic plate, endotracheal tube, stent, shunt, feeding tube and a hernia mesh.

6. The medical device of claim 1, wherein the substrate is a non-metallic substrate comprising a polymer selected from the group consisting of elastomers, natural rubbers, synthetic rubbers, silicones, polyurethanes, polyisoprene, polycarbonates, plastics, polyethylenes, polypropylenes, polyvinyl chlorides, polyvinyl alcohol, polyvinylpyrrolidones, polyvinyl acetate, polyetherketones, polyamides, polyvinylidene chloride, polystrene, polyacrylates, methancrylates, polyesters, poly(ethylene glycol) and poly(lactic acid-co-glycolic acid) and combinations thereof.

7. The medical device of claim 1, wherein the first bioactive is selected from the group consisting of an antimicrobial, antiseptic, anesthetic and an anticoagulant.

8. The medical device of claim 1, further comprising a second bioactive in a salt and a free-base form in contact with the substrate,
wherein the salt and free-base form of the second bioactive have different solubilities in an aqueous medium.

9. The medical device of claim 7, wherein the antimicrobial is selected from the group consisting of a lincosamide, clindamycin, a aminoglycoside, gentamicin, a tetracycline, minocycline, a fluoroquinolone, ciprofloxacin, a polypeptide, polymyxin, a glycopeptide, vancomycin, an aminocyclitol, spectinomycin, a macrolide, erythromycin, rifampin, a glycylcycline, tigecycline, a sulfonamide, and sulfadiazine.

10. The medical device of claim 7, wherein the antiseptic is selected from the group consisting of chlorhexidine and benzalkonium.

11. The medical device of claim 7, wherein the anesthetic is selected from the group consisting of bupivacaine, ropivacaine, lidocaine, chloroprocaine, pramoxine and mepivacaine.

12. The medical device of claim 7, wherein the anticoagulant is heparin.

13. The medical device of claim 8, wherein the first bioactive and the second bioactive are antimicrobials.

14. The medical device of claim 13, wherein the first bioactive is clindamycin and the second bioactive is minocycline.

15. The medical device in claim 8, wherein the first bioactive is an antimicrobial and the second bioactive is an antiseptic.

16. The medical device of claim 8, wherein the first bioactive is an antimicrobial and the second bioactive is an anesthetic.

17. The medical device of claim 1, wherein the substrate is a nonmetallic substrate.

18. The medical device of claim 17, wherein the first bioactive is contained within the nonmetallic substrate.

19. The medical device of claim 1 wherein the substrate is selected from the group consisting of a metal, metal alloy, glass, ceramic, and a mineral.

20. A method for manufacturing a medical device, the method comprising:
contacting a solvent with a bioactive in a salt form and a free-base form to form a solution, wherein the salt form is more soluble in an aqueous medium than is the free-base form, and
contacting a surface of the medical device with the solution,
wherein the salt and free-base form are impregnated into the medical device or are coated onto the surface of the medical device.

21. A medical device comprising:
a substrate; and
a bioactive and a salt of the bioactive in contact with the substrate, wherein the bioactive and its salt have different solubility and a different dissolution rate in a aqueous medium and wherein the bioactive is selected from the group consisting of an organic base, an organic acid and a zwitterionic compound.

22. A method for manufacturing a medical device comprising:
contacting a substrate having at least one surface with a solution comprising a solvent, a bioactive and a salt of the bioactive,
wherein the bioactive and its salt are impregnated into the medical device or coated onto the surface of the medical device in a ratio dependent on the polarity or dielectric constant of the solvent and wherein the bioactive is selected from the group consisting of an organic base, an organic acid and a zwitterionic compound.
